(19) United States
(12) Patent Application Publication
(10) Pub. No.: US 2006/0134215 A1
(43) Pub. Date: Jun. 22, 2006

(54) Film coating composition, and coating and tablet of the composition

(75) Inventors: Hiroyuki Kondo, Hamamatsu-shi (JP); Tomoyuki Shobu, Hamamatsu-shi (JP); Fujio Sekigawa, Hamamatsu-shi (JP); Toshichika Ogasawara, Tokyo (JP)

Correspondence Address: HOFFMANN & BARON, LLP, 6900 JERICHO TURNPIKE, SYOSSET, NY, 11791, US

(73) Assignee: Freund Corporation

(21) Appl. No.: 11/294,651

(22) Filed: Dec. 5, 2005

Related U.S. Application Data
(60) Provisional application No. 60/645,908, filed on Jan. 21, 2005.

(30) Foreign Application Priority Data
Dec. 6, 2004 (JP) P2004-352819

(51) Int. Cl. A61K 9/24

(52) U.S. Cl. 424/472

(57) ABSTRACT

A film coating composition comprises polyvinyl alcohol and talc, and a content of the talc is 50 to 86% by mass based on 100% by mass of the total solid components of the film coating composition. A film consists of the film coating composition, and a tablet comprises the film.

Priority is claimed on Japanese Patent Application No. 2004-352819, filed Dec. 6, 2004, and on U.S. Provisional Patent Application No. 60/645908, filed Jan. 21, 2005, the contents of which are incorporated herein by reference.

### BACKGROUND OF THE INVENTION

1. Field of the Invention

The present invention relates to a film coating composition which is usable for a film coating of a tablet, a coating which consists of the composition, and a tablet which has the coating.

2. Description of the Related Art

In order to alleviate bitterness, reduce release of an odor, shield against oxygen, provide moisture-proofing, and the like, a film coating is provided on a tablet by using a high polymer compound in general. As the high polymer compound, polyvinyl alcohol which is excellent in moisture-proofing ability and shielding ability against oxygen has been noted. However, thread-forming property of polyvinyl alcohol solution is strong, and therefore, it is difficult to conduct spray coating using an aqueous solution wherein polyvinyl alcohol is included. Furthermore, polyvinyl alcohol has high tackiness, and therefore, there is the problem that tablets adhere to each other.

Known examples of a film coating composition which has excellent film coating ability and reduced tackiness include a film coating composition which comprises polyvinyl alcohol and water-soluble polyoxyethylene glycols such as polyethylene glycol and the like (please refer to Japanese Unexamined Patent application, First Publication No. 8-59512), and a film coating composition which comprises polyvinyl alcohol, a plasticizer which is polyethylene glycol or glycerol, and 9 to 45% by mass of talc based on the composition (please refer to Published Japanese translation No. 2004-509339 of PCT).

However, the film coating composition disclosed in Japanese Unexamined Patent application, First Publication No. 8-59512 comprises polyethylene glycol and the like. Therefore, the film composition tends to transmit an odorous component included in a tablet, and therefore the shielding ability against the odorous component is poor. Furthermore, there is concern about an interaction between the medicine or food contained in a tablet and the film coating composition. Accordingly, the use of the film coating composition for tablets is restricted. The film coating composition described in Published Japanese translation No. 2003-509339 of PCT comprises a plasticizer therein. Accordingly, the shielding effect against an odorous component is poor, and there is a concern about an interaction between the medicine or food contained in a tablet and the film coating composition. Accordingly, the use of the film coating composition for tablets is restricted.

### SUMMARY OF THE INVENTION

A film coating composition of the present invention comprises polyvinyl alcohol and talc, and a content of the talc is 50 to 86% by mass based on 100% by mass of the total solid components of the film coating composition.

Furthermore, it is preferable that the film coating composition of the present invention does not comprise a plasticizer.

The film coating composition of the present invention may preferably comprise two or more kinds of polyvinyl alcohols, wherein at least one of the polyvinyl alcohols is a polyvinyl alcohol having a polymerization degree of 1000 or less, and the ratio of said polyvinyl alcohol having a polymerization degree of 1000 or less is 50% by mass or less based on the total amounts of polyvinyl alcohols (100% by mass) of the coating composition.

The film coating composition of the present invention may preferably further comprise fatty acid esters, and the content of the fatty acid esters is 25% by mass or less based on the solid contents (100% by mass) of the film coating composition.

A film of the present invention consists of the film coating composition of the present invention.

A tablet of the present invention comprises the film of the present invention.

### DETAILED DESCRIPTION OF THE INVENTION

Objects of the present invention are to provide a film coating composition, a film obtained therefrom, and a tablet which includes the film, in which, even if a plasticizer is not used, tackiness thereof can be improved, excellent film coating ability is achieved, and an oxygen shielding effect is excellent, and an odorous component in the tablet can be sufficiently shielded against.

Due to the use of the film coating composition and the film of the present invention, the odor of the odorous component can be shielded against sufficiently, the oxygen shielding effect can be achieved, tackiness of the film coating composition and the film can be improved and therefore the film coating ability of the film coating composition is excellent, even if a plasticizer is not used.

Examples of a polyvinyl alcohol which can be used in the present invention include polyvinyl alcohols which can be used for a film coating of pharmaceuticals. Among them, polyvinyl alcohols (fully hydrolyzed material) and/or polyvinyl alcohols (partially hydrolyzed material) defined by Japanese Pharmaceutical Excipient (JPE) are preferable. Polyvinyl alcohol which is a partially hydrolyzed material having a polymerization degree of 300 to 2400 and has a hydrolyzation degree of 78 to 96% by mole is more preferable. Examples of commercially available polyvinyl alcohols useful in the present invention include a variety of POVALs manufactured by Kurary Co., Ltd and a variety of GOHSENOLs manufactured by Nippon Synthetic Chemical Industry Co., Ltd. Polyvinyl alcohols which can be used in the present invention may be used singly, or in combination of two or more.

The content of the polyvinyl alcohol is preferably 14 to 50% by mass, more preferably 25 to 50% by mass, and further more preferably 29 to 49% by mass, and most preferably 33 to 49% by mass based on the total solid components (100% by mass) of the film coating composition. When the content of the polyvinyl alcohol is 14% by mass or more, a shielding effect against odorous components can be achieved sufficiently. When the content of the polyvinyl alcohol is 50% by mass or less, tackiness of the composition can be improved and excellent film coating ability is achieved. Here, the total solid components of the film coating composition means components which do not include volatilized components such as water and the like, which can be volatilized at the time of forming a film.

As the polyvinyl alcohol of the present invention, two or more kinds of polyvinyl alcohols can be included in the film coating composition of the present invention. In this case, it is preferable that at least one of said polyvinyl alcohols is a polyvinyl alcohol having a polymerization degree of 1000 or less. Due to the polyvinyl alcohol having a polymerization degree of 1000 or less, film coating ability of the coating composition including said polyvinyl alcohol can be superior to a coating composition which only includes polyvinyl alcohol(s) having a polymerization degree which is greater than 1000. The ratio of said polyvinyl alcohol having a polymerization degree of 1000 or less is preferably 50% by mass or less based on the total amounts of polyvinyl alcohols (100% by mass) of the coating composition, and more preferably 10% by mass or more and 50% by mass or less. When the ratio of polyvinyl alcohol having a polymerization degree of 1000 or less is 50% by mass or less, further excellent film coating ability can be achieved while shielding effects of the film obtained against the odorous component and oxygen can be sufficiently maintained.

Examples of talc usable in the present invention include talc which is usable as food or pharmaceutical additive, and a talc which is defined by the Japanese Pharmacopoeia is preferable.

The content of talc is 50 to 86% by mass, preferably 50 to 75% by mass, more preferably 51 to 71% by mass, and most preferably 51 to 67% by mass based on the total solid content (100% by mass) of the film coating composition. When the content of talc is 50% by mass or more, tackiness can be improved and film coating ability can be improved. When the content of talc is 86% by mass or less, shielding effect against an odorous component can be achieved sufficiently.

Furthermore, a comparative ratio of the talc to the polyvinyl alcohol is described below. The ratio is preferably 100 to 600 parts by mass of the talc, more preferably 100 to 300 parts by mass of the talc, furthermore preferably 102 to 250 parts by mass of the talc, and most preferably 105 to 200 parts by mass of the talc, based on the total solid content (100 parts by mass) of the polyvinyl alcohol. When the ratio of the talc to the polyvinyl alcohol is within this range, film coating ability is improved, and shielding ability against the odorous component can be achieved sufficiently.

Examples of inorganic compounds which can suppress tackiness include inorganic compounds such as silicic anhydride, hydrous silicon dioxide, and precipitated calcium carbonate, in addition to the talc. Among them, talc is the most preferable in order to suppress tackiness. Although inorganic compounds other than talc may also exhibit the effect of suppressing tackiness when the contents thereof are increased. However, when the inorganic compounds are excessively added, a surface condition of an obtained film coating tends to be rough, and then, problems tend to arise such that smoothness of the film coating deteriorates, clear printing cannot be obtained, and the like. The inorganic compounds other than talc can be used in combination with talc in so far as the inorganic compounds do not prevent the action of the talc.

The film coating composition of the present invention can comprise fatty acid esters in order to improve film coating ability without causing deterioration of an appearance of the film coating. Due to the effects of the fatty acid esters included therein, tackiness can be improved and further excellent film coating ability can be achieved. Furthermore, a film coating obtained by using fatty acid esters can show sufficient shielding effects against the odor of the odorous component and oxygen, and the surface condition of the obtained film coating is smooth.

The contents of fatty acid esters is preferably 25% by mass or less based on the total solid contents (100% by mass) of the film coating composition, and more preferably 0.5% by mass or more and 25% by mass or less.

As an organic compound which can suppress tackiness and is other than fatty acid esters, lecithin can also be used. The amounts of lecithin added may be limited to a small amount, since lecithin has a peculiar color, taste and smell.

As additives other than the aforementioned additives, other additive(s) which can be used generally for a film coating can be added as required to the coating composition of the present invention. Examples of said other additives include colorants such as a dye extracted from plants, a concealing agent such as silicon dioxide, calcium carbonate, titanium oxide, and the like.

The content of said other additive(s) is preferably 20% by mass or less, more preferably 10% by mass or less, and most preferably 5% by mass or less, based on the total solid content (100% by mass) of the film coating composition.

It is preferable that the film coating composition of the present invention does not comprise a plasticizer substantially, and more preferably the composition does not comprises a plasticizer at all. The recitation of “the film coating composition of the present invention does not comprise a plasticizer substantially” means that the content of the plasticizer is within a range wherein transmission of the odorous component is not promoted by the existence of the plasticizer, and that an interaction between the plasticizer in the film coating and a medicine or food contained in a tablet does not occur. Concretely, the recitation means that the content of the plasticizer is less than 5% by mass, based on the total solid content (100% by mass) of the film coating composition.

Examples of the plasticizer which has compatibility with polyvinyl alcohol include: polyethylene glycol, polyoxyethylene polyoxypropylene glycol, polyethylene glycol ether, propylene glycol, diethylene glycol, and glycerol.

The coating of the present invention can be formed by adding the film coating composition of the present invention to water to form a coating liquid, and then coating the coating liquid on a substrate using a coating machine.

The concentration of the solid content of the film coating composition is preferably 3 to 40% by mass, and more preferably 5 to 30% by mass.

Examples of the coating machine include a perforated pan type coating machine, a fluidized bed coating machine, and a conventional pan type coating machine.

A tablet of the present invention comprises the coating of the present invention. The tablet of the present invention may be a tablet wherein the coating of the present invention is provided on another coating of tablet, wherein said another coating consists of an enteric polymer compound and/or the like. In contrast, the tablet of the present invention may be a tablet wherein another coating consisting of enteric polymer compound and/or the like is further provided on the coating of the present invention provided on the tablet.

Examples of a base tablet include food or pharmaceutical products, and those may include additives which can be added if required. The present invention is particularly effective when the base tablet includes a component having an odor. Examples of the component having an odor include garlic oil, cinnamon oil, eicosapentaenoic acid (EPA), docosapentaenoic acid (DPA), docosa-hexaenoic acid (DHA), menthol, mustard oil, component having Japanese horseradish (wasabi) flavor, hydrochloric acid ethyl cysteine, and the like.

The present invention is very effective when a base tablet includes a component which deteriorates due to oxidation. Examples of the components which deteriorate due to oxidation include: fats and oils such as docosa-hexaenoic acid (DHA), eicosapentaenoic acid (EPA), linolic acid, and linolenic acid; vitamins such as lycopene, carotene, thiamine hydrochloride, riboflavin, cyanocobalamin, and ascorbic acid; and amine acids such as valine, arginine, leucine, and isoleucine.

The amount of the coating consisting of the film coating composition is generally about 2 to 30 parts by mass based on the amount of the base tablet (100 parts by mass).

The amount of talc included in the film coating composition of the present invention described above is 50% by mass or more based on the total solid component (100% by mass) of the film coating composition. Therefore, thread-forming property of polyvinyl alcohol can be suppressed, and as a result, coating can be possible using a coating liquid comprising the film coating composition of the present invention, even if a plasticizer is not used. Furthermore, since the amount of the talc is 50% by mass or more based on total solid component (100% by mass) of the film coating composition, tackiness of the coating obtained can be suppressed to prevent adhesion between tablets.

Further, when the amount of the talc is 50 to 86% by mass based on the total solid content (100% by mass) of the film coating composition, an odor of an odorous component of the tablet can be fully shielded against. Furthermore, when a component(s) having a flavoring effect and/or an odor improving effect is blended in a base tablet, and the base tablet is used for forming a chewable tablet which breaks down and dissolves readily within the mouth, reduction of the component during storage can be suppressed, and therefore, the aforementioned effect due to the component of the tablet can be continued in the mouth for a long time. Furthermore, the coating of the present invention comprises polyvinyl alcohol, the coating has an excellent oxygen shielding effect and moisture-proofing ability, and therefore, long-term storage of the tablet is possible.

### EXAMPLE

### Example 1

3 parts by mass (50% by mass based on the total solid content) of polyvinyl alcohol wherein polymerization degree is 1700 and hydrolyzation degree is 87 to 89% by mole (hereafter, this polyvinyl alcohol is described as PVA-1700) and 3 parts by mass (50% by mass based on the total solid content) of a talc (VICTORY LIGHT, manufactured by Shokozan Mining Co., Ltd.) were added to 94 parts by mass of water, and stirred while heating to 60° C. to prepare a coating liquid.

In order to evaluate the shielding ability of coatings against an odorous component, base tablets having an odor (hereafter, it is described as a base tablet having an odor) were prepared.

Base tablet having an odor: Silicon dioxide was adsorbed with garlic oil (food additive), and then, tablets were formed directly by using the adsorbed silicon dioxide and a mixed powder comprising lactose, cornstarch, microcrystalline cellulose and magnesium stearate (diameter of the tablet: 9 mm, curvature radius: 10 R).

330 g of the base tablets having an odor were placed in a perforated pan type coating machine (HI-COATER LABO, manufactured by Freund Corporation), and a coating liquid was sprayed onto the base tablets, so that the amount of the polyvinyl alcohol in the dried coating became 3 parts by mass, based on the total amount of the base tablets (100 parts by mass). Coating ability, spraying speed, operation time and a shielding effect of the base tablet having an odor on which a film coating is formed are shown in Table 1.

### Example 2

3 parts by mass (33% by mass based on the total solid content) of PVA-1700 and 6 parts by mass (67% by mass based on the total solid content) of a talc were added to 91 parts by mass of water, and stirred while heating to 60° C. to prepare a coating liquid. Coating using the coating liquid of Example 2 was conducted in the same manner as Example 1. Coating ability, spraying speed, operation time and the shielding effect of the base tablet having an odor on which a film coating is formed are shown in Table 1.

### Example 3

3 parts by mass (20% by mass based on the total solid content) of PVA-1700 and 12 parts by mass (80% by mass based on the total solid content) of a talc were added to 85 parts by mass of water, and stirred while heating to 60° C. to prepare a coating liquid. Coating using the coating liquid of Example 3 was conducted in the same manner as Example 1. Coating ability, spraying speed, operation time and the shielding effect of the base tablet having an odor on which a film coating is formed are shown in Table 1.

### Example 4

3 parts by mass (14% by mass based on the total solid content) of PVA-1700 and 18 parts by mass (86% by mass based on the total solid content) of a talc were added to 79 parts by mass of water, and stirred while heating to 60° C. to prepare a coating liquid. Coating using the coating liquid of Example 4 was conducted in the same manner as Example 1. Coating ability, spraying speed, operation time and the shielding effect of the base tablet having an odor wherein a film coating is formed are shown in Table 1.

### Example 5

2 parts by mass of PVA-1700 and 1 part by mass of polyvinyl alcohol wherein polymerization degree was 500 and hydrolyzation degree was 87 to 89% by mole (hereafter, this polyvinyl alcohol is described as PVA-500) (the total content of polyvinyl alcohols was 20 by mass based on the total solid content, and the contents of PVA-500 was 33% by mass based on the total content of polyvinyl alcohol) and 12 parts by mass (80% by mass based on the total solid content) of a talc were added to 85 parts by mass of water, and stirred while heating to 60° C. to prepare a coating liquid. Coating using the coating liquid of Example 5 was conducted in the same manner as Example 1. Coating ability, spraying speed, operation time and the shielding effect of the base tablet having an odor on which a film coating is formed are shown in Table 1.

### Example 6

1.5 parts by mass of PVA-1700 and 1.5 parts by mass of PVA-500 (the total content of polyvinyl alcohols was 20 by mass based on the total solid content, and the contents of PVA-500 was 50% by mass based on the total content of polyvinyl alcohol) and 12 parts by mass (80% by mass based on the total solid content) of a talc were added to 85 parts by mass of water, and stirred while heating to 60° C. to prepare a coating liquid. Coating using the coating liquid of Example 6 was conducted in the same manner as Example 1. Coating ability, spraying speed, operation time and the shielding effect of the base tablet having an odor on which a film coating is formed are shown in Table 1.

### Example 7

2 parts by mass of PVA-1700 and 1 part by mass of PVA-500 (the total content of polyvinyl alcohols was 18 parts by mass based on the total solid content, and the content of PVA-500 was 33% by mass based on the total content of polyvinyl alcohol) and 12 parts by mass (73% by mass based on the total solid content) of a talc and 1.5 parts by mass (9% by mass based on the total solid content) of a sucrose fatty acid ester (DK ESTER F-70 manufactured by Dai-ichi Kogyo Seiyaku Co., Ltd.) were added to 83.5 parts by mass of water, and stirred while heating to 60° C. to prepare a coating liquid. Coating using the coating liquid of Example 7 was conducted in the same manner as Example 1. Coating ability, spraying speed, operation time and the shielding effect of the base tablet having an odor on which a film coating is formed are shown in Table 1.

### Comparative Example 1

3 parts by mass (59% by mass based on the total solid content) of PVA-1700 and 2.1 parts by mass (41% by mass based on the total solid content) of a talc were added to 94.9 parts by mass of water, and stirred while heating to 60° C. to prepare a coating liquid. Coating using the coating liquid of Comparative Example 1 was conducted in the same manner as Example 1. Coating ability, spraying speed, operation time and the shielding effect of the base tablet having an odor on which a film coating is formed are shown in Table 1.

### Comparative Example 2

1 part by mass of PVA-1700 and 2 part by mass of PVA-500 (the total content of polyvinyl alcohols was 59% by mass based on the total solid content, and the content of PVA-500 was 67% by mass based on the total content of polyvinyl alcohols) and 2.1 parts by mass (41% by mass based on the total solid content) of a talc were added to 94.9 parts by mass of water, and stirred while heating to 60° C. to prepare a coating liquid. Coating using the coating liquid of Comparative Example 2 was conducted in the same manner as Example 1. Coating ability, spraying speed, operation time and the

shielding effect of the base tablet having an odor on which a film coating is formed are shown in Table 1.

### Comparative Example 3

3 parts by mass (47.6% by mass based on the total solid content) of PVA-1700, 2.7 parts by mass (42.7 by mass based on the total solid content) of a talc, and 0.6% by mass of polyethylene glycol 6000 (MACROGOL 6000, manufactured by Sanyo Chemical Industries, Ltd.) (hereafter, described as PEG6000.) were added to 93.7 parts by mass of water, and stirred while heating to 60° C. to prepare a coating liquid. Coating for the coating liquid of Comparative Example 3 was conducted in the same manner as Example 1. Coating ability, spray speed, operation time, and the shielding effect of the base tablet having an odor on which a film coating is formed are shown in Table 1.

### Comparative Example 4

As a polymer compound which was different from a polyvinyl alcohol, hydroxypropyl methylcellulose (TC-5 (RW) manufactured by Shin-Etsu Chemical Co., Ltd., which is hereinafter described as HPMC) was prepared.

6 parts by mass of HPMC was added to 94 parts by mass of water, and stirred to prepare a coating liquid.

Coating for the coating liquid of Comparative Example 4 was conducted in the same manner as Example 1. That is, the coating liquid was sprayed onto the base tablets, so that the amount of the HPMC in the dried coating became 3 parts by mass, based on the total amount of the base tablets (100 parts by mass). Coating ability, spray speed, operation time, and the shielding effect of the base tablet having an odor on which a film coating is formed are shown in Table 1.

TABLE 1

| | Example | | | | | | | Comparative Example | | | |
|---|---|---|---|---|---|---|---|---|---|---|---|
| (Parts by mass) | 1 | 2 | 3 | 4 | 5 | 6 | 7 | 1 | 2 | 3 | 4 |
| PVA-1700 | 3.0 | 3.0 | 3.0 | 3.0 | 2.0 | 1.5 | 2.0 | 3.0 | 1.0 | 3.0 | |
| PVA-500 | | | | | 1.0 | 1.5 | 1.0 | | 2.0 | | |
| HPMC | | | | | | | | | | | 6.0 |
| Talc | 3.0 | 6.0 | 12.0 | 18.0 | 12.0 | 12.0 | 12.0 | 2.1 | 2.1 | 2.7 | |
| PEG6000 | | | | | | | | | | 0.6 | |
| Sucrose fatty acid ester | | | | | 1.5 | | | | | | |
| Water | 94.0 | 91.0 | 85.0 | 79.0 | 85.0 | 85.0 | 83.5 | 94.9 | 94.9 | 93.7 | 94.0 |
| Coating ability | ∘ | ∘ | ∘ | ∘ | ∘ | ∘ | ∘ | x | ∘ | ∘ | ∘ |
| Spray speed (g/min) | 1.5 | 2.3 | 3.0 | 3.0 | 3.0 | 3.0 | 3.0 | Smaller | 2.0 | 3.0 | 3.0 |
| | | | or more | or more | or more | or more | or more | than | | | or more |
| | | | | | | | | 1.0 | | | |
| Operation time (min) | 300 | 200 | 140 | 120 | 125 | 120 | 120 | — | 250 | 150 | 70 |
| Shielding effect against | ∘ | ∘ | ∘ | ∘ | ∘ | ∘ | ∘ | — | x | x | x |
| odorous component | (None) | (None) | (None) | (None) | (None) | (None) | (None) | | (5 people) | (6 people) | (6 people) |

In Table 1, coating ability was evaluated such that an unpreferable case wherein picking and blocking of tablets occurred in spite of using the minimum spray speed (1 g/min) was represented as “×”, and a preferable case wherein such picking and blocking of a tablet did not occur in spite of using such a spray speed was represented as “∘”. Regarding the shielding effect against odor of odorous component, sensory tests were conducted by six volunteers, and the effect was evaluated such that a unpreferable case wherein five or more people felt odor was represented as “×”, and a preferable case wherein one or less people felt odor was represented as “∘”.

(1) Film Coating Ability

In the cases where polyvinyl alcohol was used, coatings of Comparative Example 1, wherein the content of talc is 41% by mass based on the total solid content of the film coating composition, was not able to be carried out. On the other hand, coatings of Examples 1 to 7, wherein the content of talc was 50 to 86% by mass based on the total solid content, were able to be carried out.

(2) Shielding Effect Against Odorous Component

None of the volunteers evaluated that the odor was sensed regarding tablets, wherein film coatings of Examples 1 to 7 were provided on the base tablets having an odor. On the other hand, five or more volunteers evaluated that the odor was sensed regarding tablets wherein film coatings of Comparative Examples 2 to 4 were provided on the base tablets having an odor, and regarding tablets in which a film coating was not provided on the base tablet.

(3) Film Coating Ability Evaluated by a Production Machine

Tablets were formed directly by using a mixed powder consisting of lactose, corn starch, and magnesium stearate (diameter of the tablet: 8 mm, radius of the tablet: 10 R), and then 50 kg of the base tablets were placed in a perforated pan type coating machine (HI-COATER 100N, manufactured by Freund Corporation), and each coating liquid of Examples 3, 5 and 7 was sprayed onto the base tablets, so that the amount of the polyvinyl alcohol in the dried coating became 3 parts by mass, based on the total amount of the base tablets (100 parts by mass). Spraying speed, and operation time are shown in Table 2.

TABLE 2

| | Example 3 | Example 5 | Example 7 |
|---|---|---|---|
| Spraying speed (g/min) | 90 | 110 | 140 |
| Operation time (min) | 540 | 460 | 360 |

As shown in the Table, film coating ability can be further improved due to use of a polyvinyl alcohol having a polymerization degree of 1000 or less as in Example 5, and a sucrose fatty acid ester as in Example 7.

(4) Oxygen Shielding Effect

Base tablets which deteriorate due to oxidation as those described below (hereinafter, described as oxidation deterioration tablets) were prepared in order to evaluate an oxygen shielding effect.

Oxidation deterioration tablets: Dextrin was adsorbed with linolic acid, and then, tablets were formed directly by using the adsorbed dextrin and a mixed powder consisting of sorbitol, microcrystalline cellulose, silicon dioxide and magnesium stearate (diameter of the tablet: 8 mm, radius of the tablet: 10 R).

330 g of the oxidation deterioration tablets were placed in a perforated pan type coating machine (HI-COATER LABO, manufactured by Freund Corporation), and each coating liquid of Examples 3 and 5 and Comparative Example 2 and 3 was sprayed onto the base tablets, so that the amount of the polyvinyl alcohol in the dried coating became 3 parts by mass, based on the total amount of the base tablets (100 parts by mass). The results of spraying speed and operation time were the same as those in Table 1.

The oxidation deterioration tablets, which had a coating film prepared with the coating liquids of Example 3 or 5 or Comparative Example 2 or 3, and oxidation deterioration tablets having no coating film were placed under aeration conditions in a thermostatic apparatus in which a temperature therein was maintained at 40° C. Then, the peroxide values of linolic acid included in the oxidation deterioration tablets, which varied with the passage of time, were determined. Then, each period which was required for the peroxide values of linolic acid thereof to exceed 20 was determined for evaluation. The results are shown in Table 3. The evaluation method of peroxide value of linolic acid was conducted based on “Methods of Analysis in Health Science” edited by the Pharmaceutical Society of Japan.

TABLE 3

| | | | | Base tablet |
|---|---|---|---|---|
| | | | | having |
| | | Comparative | Comparative | no film |
| Example 3 | Example 5 | Example 2 | Example 3 | coating |
| 14 days | 14 days | 6 days | 4 days | 6 days |
| or more | or more | | | |

In Examples 3 and 5, increase in the peroxide values of linolic acid did not confirmed, and therefore the shielding effect against oxygen were excellent. On the other hand, in Comparative Example 2 and the base tablet having no film coating, the peroxide values of linolic acid exceeded 20 on the sixth day. Furthermore, in Comparative Example 3 wherein PEG6000 was added, the peroxide value of linolic acid exceeded 20 on the 4th day. The period was shorter than that of the base tablet having no film coating, and an interaction between PEG6000 and linolic acid was confirmed.

As described above, the film coating composition of the present invention is useful for a coating film of a tablet of food, health food, pharmaceuticals, and the like.

The film coating composition is particularly effective for a film coating of a tablet which includes odorous components, and also for a tablet which includes a component which is liable to cause interaction with a plasticizer.

The present invention can provide a film coating composition and a film coating wherein excellent tackiness and film coating ability thereof can be achieved even if a plasticizer is not used, odor in the tablet can be sufficiently shielded, and an excellent oxygen shielding effect can be achieved, and can also provide a stable tablet wherein tablets do not adhere to each other, odor is very small, and preservation thereof can be possible for a long period of time.

While preferred embodiments of the invention have been described and illustrated above, it should be understood that these are exemplary of the invention and are not to be considered as limiting. Additions, omissions, substitutions, and other modifications can be made without departing from the spirit or scope of the present invention. Accordingly, the invention is not to be considered as being limited by the foregoing description, and is only limited by the scope of the appended claims.

What is claimed is:

1. A film coating composition, wherein the film coating composition comprises polyvinyl alcohol and talc, and a content of the talc is 50 to 86% by mass based on 100% by mass of the total solid components of the film coating composition.

2. The film coating composition according to claim 1, wherein the film coating composition does not comprise a plasticizer.

3. The film coating composition according to claim 1, wherein the film coating composition comprises two or more kinds of polyvinyl alcohols, and at least one of the polyvinyl alcohols is a polyvinyl alcohol having a polymerization degree of 1000 or less, and the ratio of the polyvinyl alcohol having a polymerization degree of 1000 or less is 50% by mass or less based on 100% by mass of the total amounts of the polyvinyl alcohols of the coating composition.

4. The film coating composition according to claim 1, wherein the film coating composition further comprises fatty acid esters, and the content of the fatty acid esters is 25% by mass or less based on the solid contents (100% by mass) of the film coating composition.

5. A film which consists of the film coating composition according to any one of claims 1 to 4.

6. A tablet having the film according to claim 5.

7. The film coating composition according to claim 1, wherein the polyvinyl alcohol is a partially hydrolyzed material having a polymerization degree of 300 to 2400 and has a hydrolyzation degree of 78 to 96% by mole.

8. The film coating composition according to claim 1, wherein the content of talc is 50 to 75% by mass based on 100% by mass of the total solid components of the film coating composition.

9. The film coating composition according to claim 1, wherein the content of talc is 51 to 71% by mass based on 100% by mass of the total solid components of the film coating composition.

10. The film coating composition according to claim 1, wherein the content of talc is 51 to 67% by mass based on 100% by mass of the total solid components of the film coating composition.

11. The film coating composition according to claim 1, wherein the content of the polyvinyl alcohol is 14 to 50% by mass based on 100% by mass of the total solid components of the film coating composition.

12. The film coating composition according to claim 1, wherein the content of the polyvinyl alcohol is 25 to 50% by mass based on 100% by mass of the total solid components of the film coating composition.

13. The film coating composition according to claim 1, wherein the content of the polyvinyl alcohol is 29 to 49% by mass based on 100% by mass of the total solid components of the film coating composition.

14. The film coating composition according to claim 1, wherein the content of the polyvinyl alcohol is 33 to 49% by mass based on 100% by mass of the total solid components of the film coating composition.

15. The film coating composition according to claim 1, wherein the film coating composition comprises 20% by mass or less of an additive based on 100% by mass of the total solid components of the film coating composition.

16. The film coating composition according to claim 1, wherein the film coating composition comprises a plasticizer which is less than 5% by mass based on 100% by mass of the total solid components of the film coating composition.

17. The film coating composition according to claim 1, wherein the film coating composition provides a film coating for a medicine or food

18. The tablet according to claim 6, wherein a coating consisting of an enteric polymer compound is provided at at least one of, between the film coating and a base tablet, and on the surface of the film coating of the tablet.

19. The tablet according to claim 6, wherein a base tablet of the tablet comprises an odorous component.

20. The tablet according to claim 6, wherein a base tablet of the tablet comprises a component which deteriorates due to oxidation.
